(19) United States
(12) Patent Application Publication
(10) Pub. No.: US 2024/0197460 A1
(43) Pub. Date: Jun. 20, 2024

(54) A SURGICAL DEVICE

(71) Applicant: Marsal Innovation Pty Ltd, Canterbury, Victoria (AU)

(72) Inventor: Salwan Hamoudi Musa Al-Salihi, Sydney (AU)

(21) Appl. No.: 18/556,878

(22) PCT Filed: Apr. 29, 2022
(86) PCT No.: PCT/AU2022/050399

§ 371 Date: Oct. 23, 2023

(30) Foreign Application Priority Data
Apr. 29, 2021 (AU) 2021901261

(51) Int. Cl. A61F 2/00; A61B 17/42

(52) U.S. Cl. CPC A61F 2/0045; A61B 17/42; A61B 2017/4225; A61F 2002/0081; A61F 2220/0008; A61F 2220/0033; A61F 2250/0007; A61F 2250/001; A61F 2250/0026; A61F 2250/0036; A61F 2250/006

(57) ABSTRACT

The present disclosure relates to a devices and methods for use in surgery. More particularly, the present disclosure is directed to devices and methods for the treatment of pelvic organ prolapse conditions, and in particular for the treatment of uterine prolapse.

### CROSS-REFERENCE TO RELATED APPLICATIONS

The present application is a 371 application of PCT/AU2022/050399, filed 29 Apr. 2022, which claims priority from Australian Provisional Patent Application No. 2021901261 filed on 29 Apr. 2021, the contents of which are incorporated herein by reference in their entirety.

### TECHNICAL FIELD

The present disclosure relates to a devices and methods for use in surgery. In particular, the present disclosure is directed to devices and methods for the treatment of pelvic organ prolapse conditions, and in particular for the treatment of uterine prolapse.

### BACKGROUND

Pelvic organ prolapse generally occurs when organs or other structures within the pelvic cavity descend from their normal anatomical positions. For example, uterine prolapse can occur when pelvic floor muscles and ligaments stretch and weaken, providing inadequate support for the uterus, leading to the uterus descending into and/or protruding from the vagina.

As such, pelvic organ prolapse typically occurs as a result of weakening or damage to the muscles and tissues that support the vagina, uterus and surrounding organs that otherwise function to hold them in place. Pelvic organ prolapse can affect women of any age, but is generally due to damage to supportive tissues during pregnancy and childbirth, the effects of gravity, a loss or lack of oestrogen after menopause and/or repeated straining, all of which can weaken the pelvic floor.

Various surgical treatments for pelvic organ prolapse are available depending on the type and severity thereof. Most treatments involve utilizing implantable meshes to help provide support to or suspend prolapsed organs in the pelvic cavity (i.e., mesh hysteropexy). By way of example, mesh hysteropexy allows for uterine preservation by suspending the uterus to the sacral promontory. Such procedures, however, are not without their complications including mesh exposure and mesh injuries to surrounding organs. Additionally, in cases where chronic pain, complications or the need to have a hysterectomy are involved, the removal of implanted mesh can be difficult given its ability to interact and integrate with the surrounding pelvic tissues after placement.

Accordingly, there remains a need for improved surgical devices and methods for treating pelvic organ prolapse, such as uterine prolapse, in a patient in need thereof.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure is broadly directed to a surgical device, and more particularly an implant, for assisting a surgeon during surgery, for the treatment of pelvic organ prolapse conditions, and in particular for the treatment of uterine prolapse such as hysteropexy surgery (i.e., surgical fixation of a displaced uterus), in a patient. The current disclosure is further directed to a surgical kit including said surgical device and methods of using the surgical device in hysteropexy surgery.

In a first aspect, the present disclosure provides a surgical device for performing hysteropexy in a subject, said device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject.

In particular examples, the elongate body is substantially flattened.

In some examples, the elongate body is substantially formed of a silicone or a derivative thereof.

Suitably, the elongate body comprises, consists of or consists essentially of a nonporous material. In particular examples, the elongate body does not comprise mesh.

Suitably, the first end section comprises a fastening arrangement having cooperating connecting elements at or towards respective proximal and distal end portions thereof to facilitate formation of the closed loop. In this regard, the fastening arrangement may comprise one or more openings at or towards one of the proximal or distal end portions of the first end section and adapted to receive the respective first or second free end of the elongate body therethrough and facilitate formation of the closed loop.

In some examples, the fastening arrangement is configured to be releasable to facilitate reversible formation of the closed loop.

In certain examples, the fastening arrangement is configured to facilitate adjustment of a size of the closed loop.

Suitably, the first end section further comprises one or a plurality of apertures that facilitate securing the closed loop to a portion of the subject's cervix and/or uterus. In this regard, the one or plurality of apertures can comprise one or more eyelets extending outwardly from a lateral edge of the first end section. In related examples, the first end section further comprises a flap element extending outwardly from the lateral edge thereof so as to substantially overlie the one or more eyelets.

Suitably, the first end section further comprises a textured portion disposed on an inner surface of the closed loop. In some examples, the textured portion comprises one or more channels extending along a length of the first end section.

In particular examples, the first end section further comprises a fenestrated portion configured to allow passage of fluid or exudate from an adjacent surface of the cervix and/or uterus therethrough.

In certain examples, the second end section comprises one or a plurality of further openings disposed at or towards the second free end of the elongate band that facilitate securing the device to the anterior longitudinal ligament.

In some examples, the second end section comprises one or a plurality of removable segments disposed at or towards the second free end of the elongate band.

In a second aspect, the present disclosure provides a method of performing hysteropexy in a subject, said method including the steps of:
providing a surgical device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject;
positioning the first end section adjacent the cervix and/or uterus of the subject so as to extend at least partly therearound;
forming the closed loop of the first end section around the cervix and/or uterus of the subject; and
securing the second end section to the anterior longitudinal ligament of the subject.

Suitably, the present method further comprises the step of securing the first end section to the cervix and/or uterus of the subject.

In some examples, the present method further comprises the step of making one or more incisions in a broad ligament of the subject to facilitate positioning of the first end section.

Suitably, the surgical device is that of the first aspect.

In a third aspect, the present disclosure provides a kit comprising the surgical device of the first aspect and optionally instructions for use.

Suitably, the kit is for use in the method of the second aspect.

Any embodiment herein shall be taken to apply mutatis mutandis to any other embodiment unless specifically stated otherwise.

It will be appreciated that the indefinite articles “a” and “an” are not to be read as singular indefinite articles or as otherwise excluding more than one or more than a single subject to which the indefinite article refers.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

In order that the present invention may be readily understood and put into practical effect, reference will now be made to the accompanying illustrations, wherein like reference numerals are used to refer to like elements.

FIG. 1: is a perspective view of an embodiment of a surgical device for hysteropexy surgery.

FIG. 2: is a side view of the surgical device of FIG. 1 implanted in the pelvic cavity of a subject.

FIG. 3: is an anterior view of the surgical device of FIG. 1 implanted in the subject's pelvic cavity.

FIG. 4: is a perspective view of a further embodiment of a surgical device for hysteropexy surgery.

FIG. 5: is a top down view of the surgical device of FIG. 4 in a closed position encompassing a subject's cervix.

FIG. 6: is a perspective view of another embodiment of a surgical device for hysteropexy surgery.

FIG. 7 is a cross-sectional view of the surgical device of FIG. 6.

FIG. 8 is a perspective view of a securing arrangement that can be included with the surgical devices of FIGS. 1, 4 and 6.

FIG. 9 is a top down perspective view of two different sizes of a further embodiment of a surgical device for hysteropexy surgery.

FIG. 10 is a perspective view of a further embodiment of a surgical device for hysteropexy surgery.

FIG. 11 is a side view of the surgical device of FIG. 10.

### DETAILED DESCRIPTION

The present disclosure relates to a surgical device or implant for use during hysteropexy surgery that may facilitate relatively quick and easy positioning and fixation within the pelvic cavity, whilst minimising the risk of post-surgical complications, such as erosion, exposure and trauma to the underlying cervical and/or uterine tissue.

While the principles described herein are based on methods of providing surgical devices for humans, this invention may also be extended to other mammals such as livestock (e.g. cattle, sheep), performance animals (e.g. racehorses) and domestic pets (e.g. dogs, cats), although without limitation thereto.

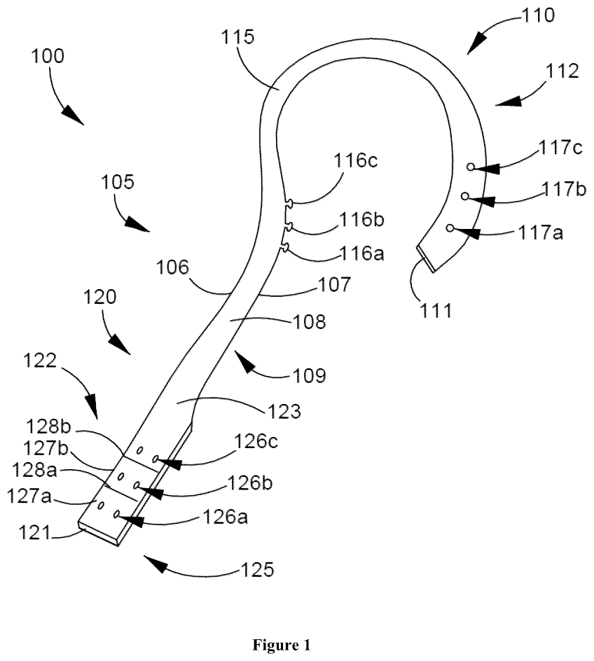

A first embodiment of an implantable surgical device 100 is provided in FIGS. 1 to 3. The device 100 includes a flexible and elongate body or band 105 that is substantially flattened and defines first and second free ends 111,121, first and second lateral edges 106,107 and inner and outer surfaces 108,109. The elongate body 105 further includes a proximally positioned first end section 110 that has a curved portion 115. The curved portion 115 is designed to encompass and contact at least a portion, such as anterior, lateral and/or posterior portions, of an outer surface of a subject's cervix 10 and optionally an adjacent lower section of their uterus 20. Suitably, the first and second lateral edges of the elongate band 105 are rounded so as to minimize the potential of compromising underlying cervical and uterine tissue.

Centrally disposed at or towards a distal end of the first end section 110 (and towards a middle segment of the elongate band 105) are three button-like projections 116a-c. As can be observed in FIG. 1, the projections 116a-c are serially spaced apart along a long axis of the inner surface 108 of the elongate band 105 and extend outwardly and perpendicularly therefrom. The first end section 110 further includes three apertures or recesses 117a-c positioned at or towards a proximal end 112 of the elongate band 110. Like the aforementioned projections 116a-c, the apertures 117a-c are disposed along a long axis of the elongate band 110 and are arranged in a similarly spaced apart fashion. The apertures 117a-c are of suitable dimensions for reversibly receiving their corresponding or respective projection 116a-c therethrough and securing the proximal end 112 of the first end section 110 to the distal end thereof.

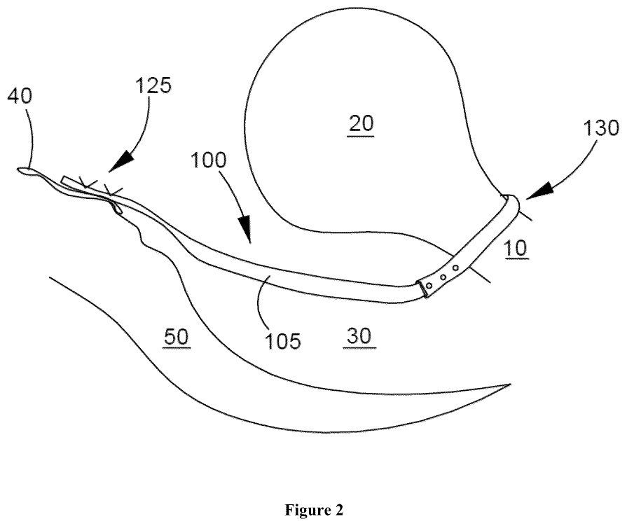

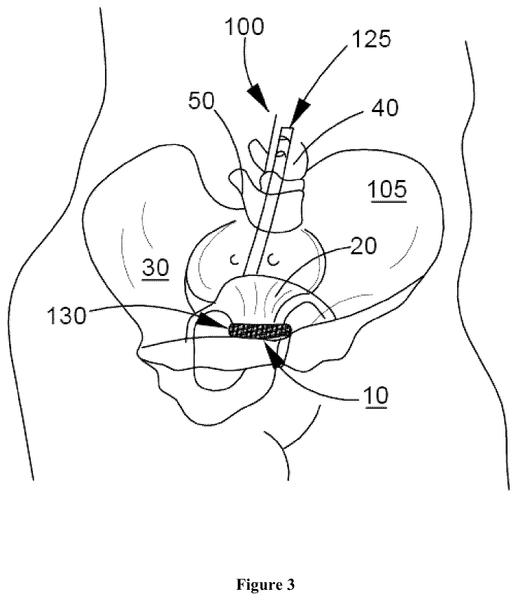

By virtue of this cooperating fastening arrangement, the curved portion 115 is reversibly or releasably operable between open and closed positions so as to be capable of forming a closed loop 130 around the cervix 10 and/or uterus 20 when appropriately positioned in the pelvic cavity 30, as shown in FIGS. 2 and 3. In the event where post-surgical complications occur or the subject subsequently requires a hysterectomy, this arrangement also allows for easier removal of the device 100 compared to that for traditional mesh-based implants. To this end, there is little or no tissue interaction between the elongate band 105 and the surrounding tissue compared to that for mesh-based implants (e.g., no collagen deposition and no in-growth of tissue around the device 100 unlike mesh-based implants of the prior art).

It is envisaged that the respective positioning of the apertures 117a-c and the projections 116a-c in the elongate band 110 may also be reversed without influencing the functionality of the device 100. Moreover, it will be understood that any number of apertures 116a-c and their corresponding projections 117a-c (e.g., 1, 2, 3, 4, 5, 6, 7, etc) may be utilized for the present invention to facilitate formation of the closed loop 130.

Additionally, the skilled person will appreciate that alternative fastening or securing arrangements as are known in the art may also be used for the devices 100,200,300,400,500 described herein, such as a button snap arrangement, a button hole or slit arrangement, a lasso-like arrangement, a buckle clasp arrangement and loop fasteners. Additionally, the fastening arrangement suitably provides different or alternative loop forming engagement or securement locations, which are selectable by the surgeon depending upon the anatomical size of the subject's cervix and/or uterus or other anatomical or surgical considerations. By way of example, each of the projections 116a-c may be engaged to a different aperture 117a-c at a particular position along the elongate band 105 so as to alter the size of the closed loop 130 as required by a surgeon.

Referring to FIGS. 2 and 3, the second end section 120 of the elongate band 105 is adapted to be secured to the anterior longitudinal ligament (ALL) 40, which runs along the anterior surface of the vertebral bodies and intervertebral discs of the spine 50. In this regard, the second end section 120 includes a tapered portion 123 that widens to form a securable portion 125 at or towards a distal end 122 of the elongate band 105. In the embodiment provided, the securable portion 125 provides three pairs of adjacent suture openings or slits 126a-c, with each pair serially spaced apart from each other along a long axis of the elongate band 105. Each pair of suture openings 126a-c is dimensioned to receive one or more sutures (or other fastening element known in the art) therethrough to connect the securable portion 125 to the ALL 40.

The device 100 is also configured to allow for altering a length of the second end section 120. To this end, the securable portion 125 further includes a pair of removable segments 127a-b positioned serially at the second free end 121 of the elongate band 105. As shown in FIG. 1, the removable segments 127a-b are defined or delineated by respective visible indicators 128a-b extending transversely across the elongate band 105. Each removable segment 127a-b also includes a single pair of the suture openings 126a-b. By virtue of this arrangement, a surgeon is able to remove or trim one or more removable segments 127a-b from the distal end 122 of the elongate band 105 to alter or adjust a length of the securable portion 125 as required by, for example, the anatomical size of the subject in question and/or the degree of displacement of the subject's cervix 10 and uterus 20, such that these organs can be appropriately suspended or positioned in the pelvic cavity 30 upon placement of the device 100.

It will be appreciated that the surgical devices described herein may include any number (e.g., 1, 2, 3, 4, 5, 6, 7, 8 etc) of the pair of suture openings 126a-c and the removable segments 127a-b so as to facilitate suitable securement of the device to the ALL 40. Additionally, the removable segments 127a-b can be, for example, about 0.5 cm to about 5 cm long (e.g., about 0.5, 0.75, 1.0, 1.25, 1.5, 1.75, 2.0, 2.25, 2.5, 2.75, 3.0, 3.25, 3.5, 3.75, 4.0, 4.25, 4.5, 4.75 or 5.0 cm, inclusive of any range therein). Furthermore, each of the openings of the suture openings is at least about 0.5 mm, at least about 1.0 mm, at least about 1.5 mm or at least about 2.0 mm (e.g., about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 mm or any range therein) in diameter.

As used herein, the terms “approximately” and “about” refer to tolerances or variances associated with numerical values recited herein. The extent of such tolerances and variances are well understood by persons skilled in the art. Typically, such tolerances and variances do not compromise the structure, function and/or implementation of the devices and methods described herein.

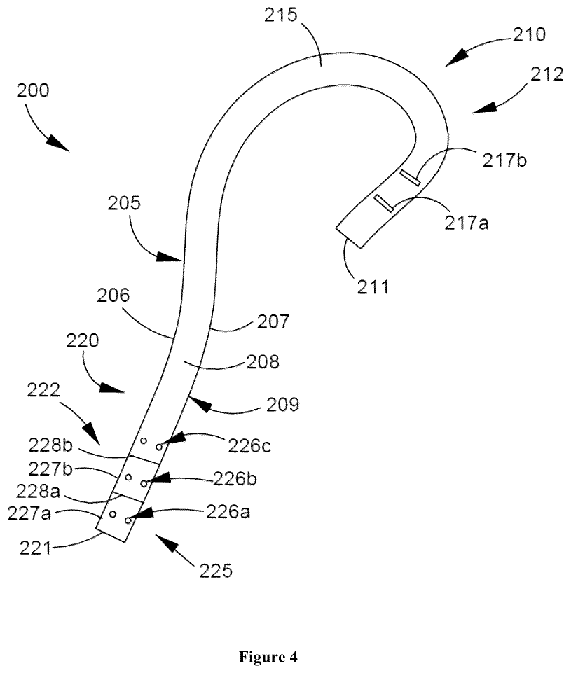

A further embodiment of a surgical device 200 of the invention is provided in FIG. 4. The surgical device 200 is similar to the previously described device 100 and includes a flexible, flattened and elongate body 205 that defines first and second free ends 211,221, first and second lateral edges 206,207 and inner and outer surfaces 208,209 thereof. Again, the elongate body 205 defines a proximally positioned first end section 210 that includes a curved portion 215 for contacting an outer surface of a subject's cervix 10 and/or uterus 20.

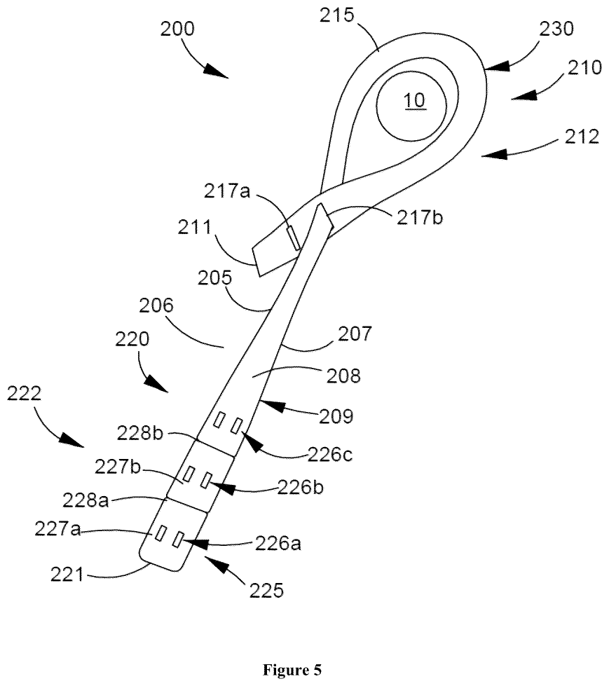

The first end section 210 includes a pair of adjacent slit-like openings 217a-b disposed in parallel and spaced along a proximal end 212 of the elongate band 210. Each of the openings 217a-b is positioned transversely across the elongate band 205 and adapted for receiving a distal end 222 of the elongate band 205 therethrough. By virtue of this fastening arrangement, the curved portion 215 can form a closed loop 230 around the subject's cervix 10 and/or uterus 20, as shown in FIG. 5. Additionally, the respective opening 217a-b that receives the distal end 222 of the elongate band 205 can be chosen by a surgeon based on the size of the closed loop 230 required to encompass the subject's cervix 10 and/or uterus 20. To this end, it will be appreciated that the first end section 210 may include any number of adjacent slit-like openings 217a-b (e.g., 1, 2, 3, 4, 5, 6, 7, 8 etc) to facilitate achieving an appropriate length or size of the closed loop 230 for the subject in question.

This fastening arrangement also allows for the closed loop 230 to naturally tighten around the cervix 10 over time. Alternatively or additionally, the closed loop 230 may be adjusted manually by a surgeon to a size appropriate for the subject's cervix 10 and its positioning within the pelvic cavity 30.

In alternative embodiments, it is envisaged that the one or more openings 217a-b may instead be disposed at a distal end of the first end section 210 (i.e., at a middle region of the elongate band 205). Accordingly, in such an example, the proximal end 212 of the elongate band 205 may instead be received through the one or more openings 217a-b so as to form the closed loop 230.

For the device 200, the second end section 220 of the elongate band 205 is similarly adapted to be secured to the ALL 40. A distally positioned securable portion 225 provides three pairs of adjacent suture openings or slits 226a-c, with each pair serially spaced apart from each other along a long axis of the elongate band 205. Each pair of suture openings 226a-c is dimensioned to receive one or more sutures, staples or the like therethrough that connect the securable portion 225 to the ALL 40. The device 200 is also configured to allow for altering a length of the second end section 220, by way of a pair of removable segments 227a-b positioned serially at the second free end 221 of the elongate band 205. As shown in FIGS. 4 and 5, the removable segments 227a-b are defined or delineated by respective visible indicators 228a-b extending transversely across the elongate band 205. Each removable segment 227a-b also includes a single pair of the suture openings 226a-c.

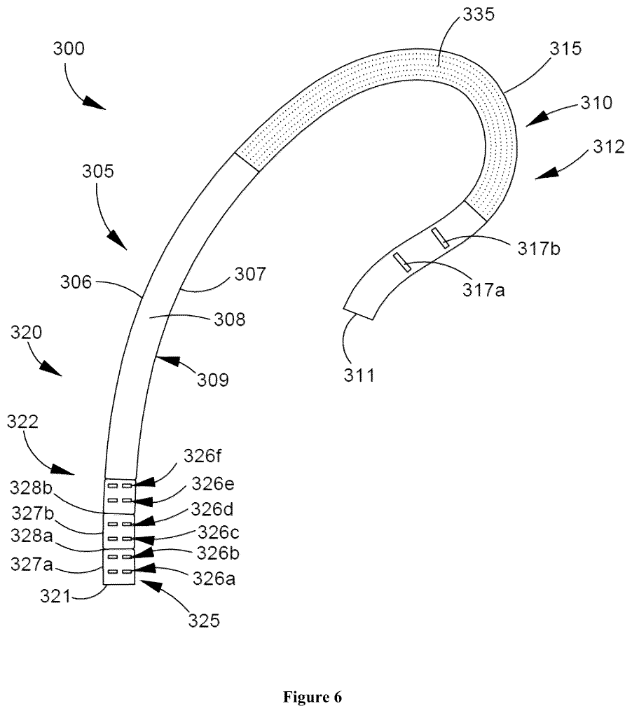

FIG. 6 provides a further embodiment of a surgical device 300 of the invention that is similar to that surgical device 200 illustrated in FIGS. 4 and 5. In brief, the surgical device 300 includes a flexible, flattened and elongate band or body 305 that defines first and second free ends 311,321, first and second lateral edges 306,307 and inner and outer surfaces 308,309 thereof. Again, the elongate body 305 defines a proximally positioned first end section 310 that includes a curved portion 315 for contacting an outer surface of a subject's cervix 10 and/or uterus 20. The first end section 310 includes a pair of adjacent elongate slits 317a-b disposed and spaced along a proximal end 312 of the elongate band 305. Each of the slits 317a-b is positioned transversely across the elongate band 305 and adapted for receiving a distal end 322 of the elongate band 305 therethrough. The second end section 320 includes a distally positioned securable portion 325, which includes six pairs of adjacent suture openings or slits 326a-f. The securable portion 325 further includes a pair of removable segments 327a-b positioned serially at the second free end 321 of the elongate band 305. The first end section 310 of the elongate band 305 comprises a porous or fenestrated portion 335 comprising a plurality of openings and configured to allow passage therethrough of a fluid or exudate from the adjacent surface of the cervix 10 and/or uterus 20.

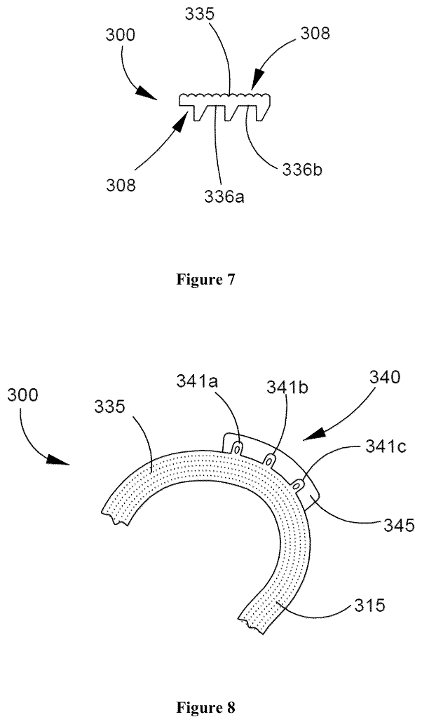

In order to minimize or prevent slipping or displacement of the devices 100,200,300,400,500 described herein off the subject's cervix 10 and onto, for example, the body of the uterus 20, the elongate band 105,205,305,405,505 may comprise a textured surface or portion, such as a high friction or patterned surface, at least partly along an inner surface of the closed loop 130,230,330,430,530. The textured surface is designed for contacting or abutting a surface of the subject's cervix 10 and/or uterus 20, particularly anterior aspects thereof, to inhibit movement of the closed loop 130,230,330,430,530 relative thereto. In a particular embodiment illustrated in FIG. 7, the fenestrated portion 335 further includes a grooved or roughened inner surface 308 having a series of channels 336a-b that extend longitudinally along the elongate band 305.

In another example provided in FIG. 8, the devices 100,200,300 described herein can further comprise a securing arrangement 340 for securing the closed loop 330 to the cervix 10 and/or uterus 20. For the purposes of illustration only, the securing arrangement 340 in FIG. 8 is described in relation to the device 300. The securing arrangement 340 includes three spaced apart loops or eyelets 341a-c positioned along the first lateral edge 306 of the first end section 310 of the elongate band 305 and extending outwardly therefrom. The eyelets 341a-c are of suitable dimensions for receiving one or more sutures, staples or other surgical fasteners therethrough and thereby facilitate securing the closed loop 330 to a surface (e.g., an anterior, lateral and/or posterior surface) of the subject's cervix 10 and/or uterus 20. In the embodiment provided, the securing arrangement 340 further comprises a flap element 345 integral with the elongate band 305 and extending outwardly from the first lateral edge 306 thereof so as to substantially overlie the three eyelets 341a-c. To this end, the flap element 345 is configured to cover, for example, any knots from sutures associated with securing the device 300 to the cervix 10 and/or uterus 20 by way of the eyelets 341a-c.

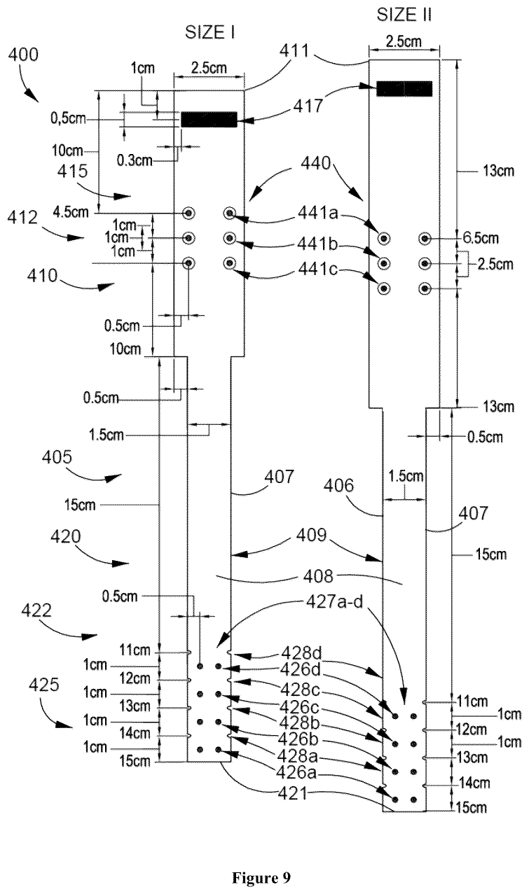

FIG. 9 demonstrates another embodiment of a surgical device 400 of the invention, for which two different sizes are illustrated (sizes I and II). The surgical device 400 is similar to surgical devices 200, 300 and includes an elongate band or body 405 that defines first and second free ends 411,421, first and second lateral edges 406,407 and inner and outer surfaces 408,409 thereof. The elongate body 405 again defines a proximally positioned first end section 410 that includes a curved or curvable portion 415 for contacting an outer surface of a subject's cervix 10 and/or uterus 20. The first end section 410 includes an adjacent elongate slit 417 disposed transversely at a proximal end 412 of the elongate band 405. The slit 417 is of suitable dimensions for receiving a distal end 422 of the elongate band 405 therethrough. From FIG. 9, the first end section 410 is of broader or wider dimensions than a second end section 420 extending distally therefrom. In this regard, the boundary between the first and second end sections 410,420 is defined by a stepped arrangement at the first and second lateral edges 406,407.

As illustrated in FIG. 9, the second end section 420 of the surgical device 400 includes a distally positioned securable portion 425, which includes four pairs of adjacent suture openings or slits 426a-d. The securable portion 425 further includes four removable segments 427a-d positioned serially at the second free end 421 of the elongate band 405. The four removable segments 427a-d are further defined by pairs of opposed notches 428a-d in the first and second lateral edges 406,407 of the elongate band 405. Although not illustrated in FIG. 9, it is further envisaged that the slit 417 and/or the adjacent suture openings 426a-d may include a thickened reinforcing portion disposed at least partly around the respective openings thereof to inhibit or prevent ripping or tearing of these structures when under tension.

In addition to the above, the surgical device 400 comprises a securing arrangement 440 for securing the closed loop 430 to the cervix 10 and/or uterus 20. The securing arrangement 440 includes three pairs of serially spaced apart openings or eyelets 441a-c positioned approximately centrally along the first end section 410 of the elongate band 405. The openings 441a-c are of suitable dimensions for receiving one or more sutures, staples or other surgical fasteners therethrough and thereby facilitate securing the closed loop 430 to a surface of the subject's cervix 10 and/or uterus 20. In the embodiment provided, the openings 441a-c include a thickened reinforcing portion or ridge disposed therearound so as to inhibit or prevent ripping of these structures when under tension.

The embodiments provided in FIG. 9 further demonstrate exemplary dimensions with respect to the surgical device 400. It will be appreciated that these dimensions are for illustration purposes only and not to be considered limiting for the illustrated embodiments. To this end, it is envisaged that various modifications may be made to those dimensions provided in FIG. 9 without departing from the scope of the present invention.

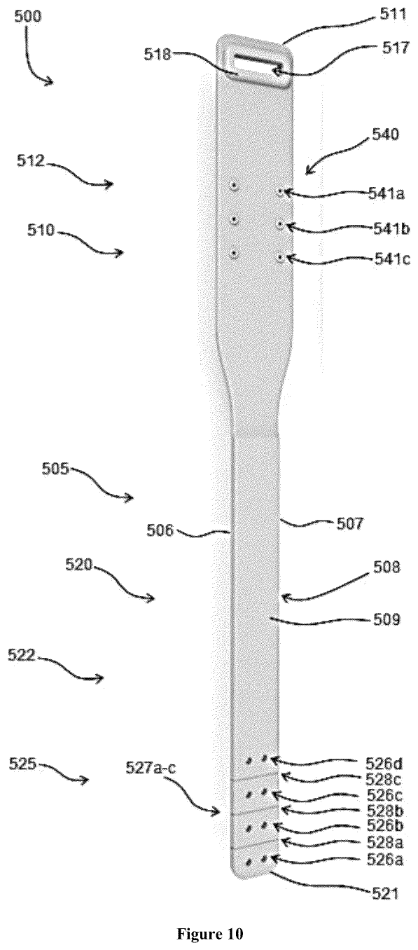

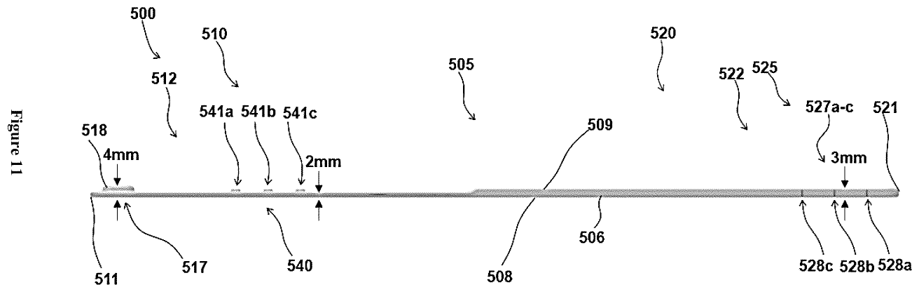

FIGS. 10 and 11 demonstrate another embodiment of a surgical device 500 of the invention. The surgical device 500 is similar to surgical devices 200, 300,400 and includes an elongate band or body 505 that defines first and second free ends 511,521, first and second lateral edges 506,507 and inner and outer surfaces 508,509 thereof. The elongate body 505 again defines a proximally positioned first end section 510 and a distally positioned second end section 520. Unlike the surgical devices 100,200,300,400, however, the first end section 510 does not include a curved portion. Rather, the first end section 510 and the second end section 520 together define the elongate band 505 as being substantially straight therebetween. Notwithstanding this, the first end section 510 is designed to contact an outer surface of a subject's cervix 10 and/or uterus 20.

The first end section 510 includes an elongate slit 517 disposed transversely at or towards a proximal end 512 of the elongate band 505. As can be observed from FIGS. 10 and 11, the slit 517 is bordered or surrounded by a thickened reinforcing portion 518 that is disposed around the respective opening thereof and extends outwardly from the outer surface 509. This arrangement is designed to inhibit or prevent ripping of the slit 517 when under tension in the pelvic cavity 40. As can be observed from FIG. 10, the thickened reinforcing portion 518 is surrounded or bordered on each side by relatively thinner portions of the first end section 510, which allows for easier foldability and provides a leading edge at the first free end 511 for smoother insertion through surgical incisions. In alternative embodiments, the first end section 510 may include a thickened portion that extends distally from the first free end 511 to just beyond the slit 517 so as to again provide reinforcement thereto.

The slit 517 is of suitable dimensions for receiving the first free end 521 and a distal end 522 of the elongate band 505 therethrough. From FIG. 10, the first end section 510 is of broader or wider dimensions than the second end section 520 extending distally therefrom. As illustrated in FIG. 11, the first end section 510 is also of thinner dimensions than the second end section 520. In this regard, the boundary between the first and second end sections 510,520 is defined by a change in thickness and a tapered neck portion at the first and second lateral edges 506,507 approximately at a middle section of the elongate band 505. The relatively increased thickness of the second end section 510 along its length thereof is intended to reduce or minimize stretching of the surgical device 500 under relative high force loads once suitably implanted within the pelvic cavity 40.

As illustrated in FIG. 10, the second end section 520 of the surgical device 500 includes a distally positioned securable portion 525, which includes four pairs of adjacent suture openings or slits 526a-d. The securable portion 525 further includes three removable segments 527a-c positioned serially at the second free end 521 of the elongate band 505. The three removable segments 527a-c are further defined by serially and equidistant positioned indented or molded guides 528a-c that extend transversely around the inner and outer surfaces 508,509 and the first and second lateral edges 506,507 of the elongate band 505 so as to function as trim guides for a user. As illustrated in FIG. 10, the adjacent suture openings 526a-d have a diameter of about 2.0 mm so as to cater for an appropriately size suture hook or needle, although any suitable diameter is envisaged (e.g., about 1.0 mm, 1.5 mm, 2.0 mm, 2.5 mm, 3.0 mm, 3.5 mm, 4.0 mm or any range therein). Additionally, and although not demonstrated in FIGS. 10 and 11, the adjacent suture openings 526a-d may include a thickened reinforcing portion disposed at least partly around the respective openings thereof to inhibit or prevent ripping of these structures when under tension.

Similar to the surgical device 400, the surgical device 500 also includes a securing arrangement 540 for securing the closed loop 530 to the cervix 10 and/or uterus 20. The securing arrangement 540 includes three pairs of serially spaced apart openings or eyelets 541a-c positioned approximately centrally along the first end section 510 of the elongate band 505. The openings 541a-c are of suitable dimensions (e.g., 0.5, 0.75, 1.0, 1.25, 1.5, 2.0, 2.25, 2.5 mm or any range therein) for receiving one or more sutures, staples or other surgical fasteners therethrough and thereby facilitate securing the closed loop 530 to a surface of the subject's cervix 10 and/or uterus 20. In the embodiment provided, the openings 541a-c include a thickened reinforcing portion or ridge disposed therearound so as to inhibit or prevent ripping of these structures when under tension.

Suitably, the surgical devices 100,200,300,400,500 described herein are integrally constructed of a medical-grade or implant-grade silicone, a derivative thereof or other suitable medical-grade or implant-grade polymer or flexible non-resorbable material (e.g., polypropylene, polyester, polyethylene, urethane, polyurethane, copolymers, and block copolymers) as are known in the art. In some embodiments, the surgical devices described herein include a reinforcing mesh, such as a fine titanium mesh, disposed or contained within the medical grade silicone or other suitable medical material that substantially makes up the device. The reinforcing mesh suitably improves the tensile strength of the surgical device, whilst also allowing for flexibility thereof. In other embodiments, the surgical device may comprise a material that is substantially transparent or translucent so as to allow for the visualization of underlying anatomic structures.

In certain embodiments, the surgical devices 100,200,300,400,500 described herein are constructed of a nonporous material, such as a nonporous medical-grade or implant-grade silicone. The term “nonporous” means that, aside from the openings for receiving sutures, the surgical device (or a substrate thereof) is substantially free of pores. To this end, the surgical device of the invention suitably does not comprise or is not made of a surgical or implantable mesh, mesh implant or mesh material (e.g., a synthetic mesh, such as a polypropylene mesh).

In particular embodiments, the elongate band described herein has a width of about 0.5 cm to about 5.0 cm (e.g., about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 cm or any range therein) between respective edges thereof. As noted above, the first and second end sections of the elongate band may be of different widths. Suitably, the first end section is wider than the second end section to increase the contact surface area of the closed loop for the subject's cervix. In particular embodiments, the first end section has a width of about 1.5 cm to about 3.5 cm, or more particularly about 2.5 cm and the second end section has a width of about 1.0 cm to about 2.0 cm, or more particularly about 1.5 cm.

Suitably, the elongate band described herein has a length of about 20 cm to about 40 cm (e.g., about 21, 22, 23, 24, 25, 26, 27, 28, 29, 30, 31, 32, 33, 34, 35, 36, 37, 38, 39, 40 cm or any range therein). In particular embodiments, the elongate band described herein has a length of about 22 cm to about 30 cm. In some embodiments, the elongate band described herein has a length of about 25 cm. In other embodiments, the elongate band described herein has a length of about 28 cm.

In particular embodiments, the first end section described herein is about 7.5 cm to about 15.0 cm in length (e.g., about 7.5, 8.0, 8.5, 9.0, 9.5, 10.0, 10.5, 11.0, 11.5, 12.0, 12.5, 13.0, 13.5, 14.5, 15.0 cm in length or any range therein). In one embodiment, the first end section is about 10 cm in length. In another embodiment, the first end section is about 13 cm in length.

In various embodiments, the second end section described herein is about 10 cm to about 20 cm in length (e.g., about 10.0, 10.5, 11.0, 11.5, 12.0, 12.5, 13.0, 13.5, 14.5, 15.0, 15.5, 16.0, 16.5, 17.0, 17.5, 18.0, 18.5, 19.0, 19.5, 20.0 cm in length or any range therein). In particular embodiments, the second end section is about 15 cm in length. In certain embodiments, the first end section is about 10 cm in length and the second end section is about 15 cm in length. In other embodiments, the first end section is about 13 cm in length and the second end section is about 15 cm in length.

Further, the elongate band described herein is preferably relatively thin, whilst maintaining suitable tensile strength, similar to that of a mesh for use in hysteropexy. In some embodiments, the elongate band described herein has a thickness of about 1.0 mm to about 5 mm (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 mm or any range therein). In particular embodiments, the elongate band described herein has a thickness of about 3.0 mm.

As noted above, the elongate band may have portions of different thickness along its length. Suitably, the first end section is thinner than the second end section to allow for improved flexibility when positioned around the subject's cervix within the pelvic cavity. In particular embodiments, the first end section has a thickness (e.g., an average thickness) of about 1.0 mm to about 3.0 mm, and the second end section has a thickness of about 2.5 mm to about 4.5 mm. In particular embodiments, the first end section has a thickness of about 1.5 mm. In other embodiments, the first end section has a thickness of about 2.0 mm. In certain embodiments, the second end section has a thickness of about 3.0 mm. In various embodiments, the second end section has a thickness of about 4.0 mm. In some embodiments, the first end section has a thickness of about 2.0 mm and the second end section has a thickness of about 3.0 mm.

For illustration purposes, the following details with respect to methods of surgical placement relate to the surgical device 100. It will be appreciated, however, that similar methods may apply for any of the embodiments of surgical device described herein. Moreover, embodiments of the below hysteropexy method may be performed using any method known in the art, such as robotically, laparoscopically, or in open surgery through a laparotomy incision.

Prior to implantation of the surgical device 100 hereinbefore described, the patient's pelvic cavity is exposed and the uterus 20 and cervix 10 are visualised. The uterus 20 is then pulled dorsally and apically so that the anterior aspect of the cervix 10 is exposed. Small incisions are then made with the use of scissors and/or cautery in the broad ligament on either side thereof and adjacent the cervix 10. Advantageously, the incisions to be made in the broad ligament are smaller and more discrete than those required for mesh hysteropexy. The curved portion 115 of the surgical device 100 can then be placed in contact with the anterior aspect of the cervix 10 (and optionally the uterus 20) with the proximal end 112 of the elongate band 105 being fed through one incision in the broad ligament and the distal end 122 of the elongate band 105 being fed through the other incision in the broad ligament.

The three projections 116a-c of the first end section 110 can then be secured or engaged to their corresponding aperture 117a-c at the proximal end 112 of the elongate band 105 so as to form a suitably dimensioned closed loop 130 surrounding the subject's cervix 10, as shown in FIGS. 2 and 3. As noted above, the size of the closed loop 130 may be altered or adjusted by securing the projections 116a-c to different apertures 117a-c as determined by the surgeon. In this regard, the join of the closed loop 130 is to be positioned in the midline of the pelvic cavity 40 and adjacent a posterior aspect of the cervix 10.

As hereinbefore described, the closed loop 130 or curved portion 115 may optionally be attached to the cervix 10, such as an anterior aspect thereof, by way of a securing arrangement, such as that illustrated in FIGS. 8 and 9, and sutures or the like.

The cervix 10 and uterus 20 are then lifted or suspended to an appropriate position within the pelvic cavity 30. One or more removable segments 127a-b may then be trimmed from the securable portion 125 so as to achieve an appropriate length of the elongate band 105 that maintains the cervix 10 in this suspended position. The securable portion 125 of the elongate band 105 is then attached to the ALL 40 at the sacral promontory of the sacral spine 50 on to the anterior longitudinal ligament by way of permanent sutures (like Gore-Tex or prolene) or other suitable fastening element. Any remaining incisions are surgically closed and the device may be covered with peritoneum as per surgical steps relevant to the procedure. It is also envisaged that additional surgical procedures, such as ovariectomy and/or burch colposuspension may be executed at the time of the aforementioned hysteropexy surgery.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

1. A surgical device for performing hysteropexy in a subject, said device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject.

2. The device of claim 1, wherein the elongate body is substantially flattened.

3. The device of claim 1, wherein the elongate body is substantially formed of a silicone or a derivative thereof.

4. The device of claim 1, wherein the first end section comprises a fastening arrangement having cooperating connecting elements at or towards respective proximal and distal end portions thereof to facilitate formation of the closed loop.

5. The device of claim 4, wherein the fastening arrangement comprises one or more openings at or towards one of the proximal or distal end portions of the first end section and adapted to receive the respective first or second free end of the elongate body therethrough and facilitate formation of the closed loop.

6. The device of claim 4, wherein the fastening arrangement is configured to be releasable to facilitate reversible formation of the closed loop.

7. The device of claim 4, wherein the fastening arrangement is configured to facilitate adjustment of a size of the closed loop.

8. The device of claim 1, wherein the first end section further comprises one or a plurality of apertures that facilitate securing the closed loop to a portion of the subject's cervix and/or uterus.

9. The device of claim 8, wherein the one or plurality of apertures comprise one or more eyelets extending outwardly from a lateral edge of the first end section.

10. The device of claim 9, wherein the first end section further comprises a flap element extending outwardly from the lateral edge thereof so as to substantially overlie the one or more eyelets.

11. The device of claim 1, wherein the first end section further comprises a textured portion disposed on an inner surface of the closed loop.

12. The device of claim 11, wherein the textured portion comprises one or more channels extending along a length of the first end section.

13. The device of claim 1, wherein the first end section further comprises a fenestrated portion configured to allow passage of fluid or exudate from an adjacent surface of the cervix and/or uterus therethrough.

14. The device of claim 1, wherein the elongate body comprises, consists of or consists essentially of a nonporous material.

15. The device of claim 1, wherein the second end section comprises one or a plurality of further openings disposed at or towards the second free end of the elongate band that facilitate securing the device to the anterior longitudinal ligament.

16. The device of claim 1, wherein the second end section comprises one or a plurality of removable segments disposed at or towards the second free end of the elongate band.

17. A method of performing hysteropexy in a subject, said method including the steps of:
providing a surgical device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject;
positioning the first end section adjacent the cervix and/or uterus of the subject so as to extend at least partly therearound;
forming the closed loop of the first end section around the cervix and/or uterus of the subject; and
securing the second end section to the anterior longitudinal ligament of the subject.

18. The method of claim 17, further comprising the step of securing the first end section to the cervix and/or uterus of the subject.

19. The method of claim 17, further comprising the step of making one or more incisions in a broad ligament of the subject to facilitate positioning of the first end section.

20. (canceled)

21. A kit comprising a surgical device for performing hysteropexy in a subject, said device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject and optionally instructions for use.

22. (canceled)
